(19) United States
(12) Patent Application Publication
(10) Pub. No.: US 2019/0216450 A1
(43) Pub. Date: Jul. 18, 2019

(54) RETRACTOR WITH ADJUSTABLE BLADES

(71) Applicant: Spineology Inc., St. Paul, MN (US)

(72) Inventors: Todd BJORK, Hudson, WI (US); Dan MCPHILLIPS, Ham Lake, MN (US)

(21) Appl. No.: 16/250,628

(22) Filed: Jan. 17, 2019

Related U.S. Application Data
(60) Provisional application No. 62/618,579, filed on Jan. 17, 2018.

(51) Int. Cl. A61B 17/02

(52) U.S. Cl. CPC A61B 17/025; A61B 2017/0256; A61B 17/0206; A61B 17/0218

(57) ABSTRACT

A retractor including length adjustable blades is provided. The adjustable blades may include a blade body and an adjustable blade portion. The length of the blades may be adjusted using any desired mechanism including but limited to a screw actuated mechanism, a spring loaded mechanism, a rack and pinion mechanism and/or a tensioning mechanism.

### PRIORITY

This application claims the benefit of U.S. Provisional Application Ser. No. 62/618,579, filed on Jan. 17, 2018, which is hereby incorporated herein by reference in its entirety.

### FIELD

The present invention generally relates to retractors. More particularly, the present invention relates to a retractor useful for creating a minimally invasive access opening to an intervertebral space.

### BACKGROUND

A variety of tools are available for surgeons for retracting tissue to create a surgical access. However, the deeper a retractor is seated into a patient, the longer the retractor blade must be in order to prevent tissue creep. Most retractors use skinny shims that slide down a channel in the retractor blade. The shims are flimsy and ineffective in stopping the tissue creep in larger patients or at larger angled trajectories. Thus, there is a continuing need to provide improved tools for retracting a patient's tissues.

### SUMMARY

The disclosure includes a retractor with an adjustable blade length so that the blade can be shortened when in the patient. As the retractor blade is articulated out, it may abut against a bony structure, such as for example, the sacrum. When the blade impacts surrounding bony structures, the whole retractor system may be moved out of alignment. By adjusting the length of the blade shorter upon articulation, this can be avoided.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 is a perspective view of a retractor with adjustable blades in accordance with certain embodiments.

FIG. 2 is top plan view of a retractor with adjustable blades in accordance with certain embodiments.

FIG. 3 is another top plan view of a retractor with adjustable blades in accordance with certain embodiments.

FIG. 4 is front elevational view of a retractor with adjustable blades in accordance with certain embodiments.

FIG. 5 is a perspective view of a retractor with adjustable blades in accordance with certain embodiments wherein the frame is in an opened position.

FIG. 6 is a front elevational view of a retractor with adjustable blades in accordance with certain embodiments wherein the blades are in a toed position.

FIG. 7 is a perspective view of a retractor with adjustable blades, including a plurality of medial blades, in accordance with certain embodiments.

FIG. 8 is a perspective view of a retractor with adjustable blades, including a medial blade, in accordance with certain embodiments.

FIG. 9 is a detailed perspective view of a retractor with adjustable blades in accordance with certain embodiments wherein detail of the attachment of a medial blade is depicted.

FIG. 10 is a side view of the retractor medial blade holder component depicted in FIG. 9.

FIG. 11 is an end view of the retractor frame component depicted in FIG. 9.

FIGS. 12-13 are perspective views of a blade of the retractor with adjustable blades in an up or contracted position in accordance with certain embodiments.

FIGS. 14-15 are perspective views of a blade of the retractor with adjustable blades in a down or extended position in accordance with certain embodiments.

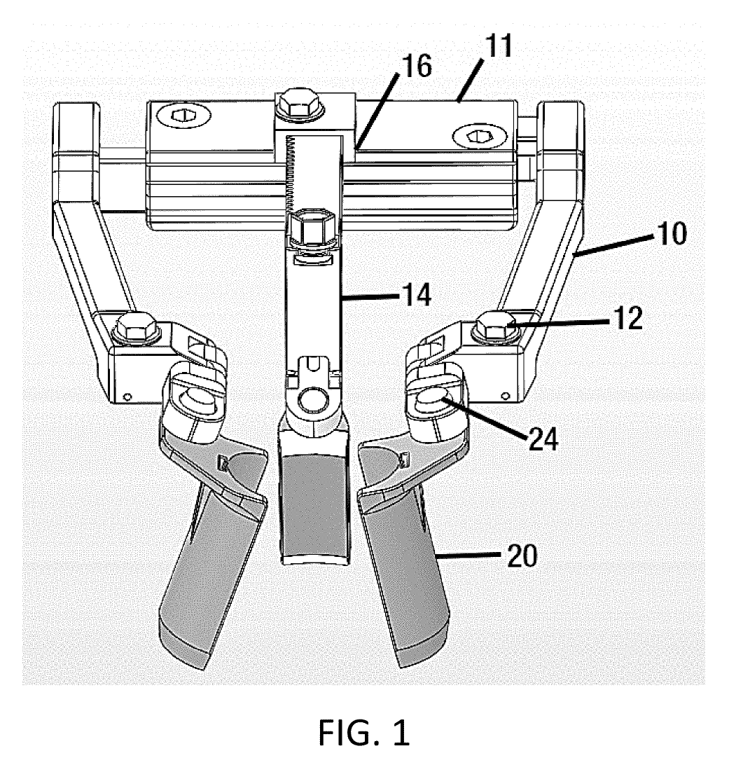

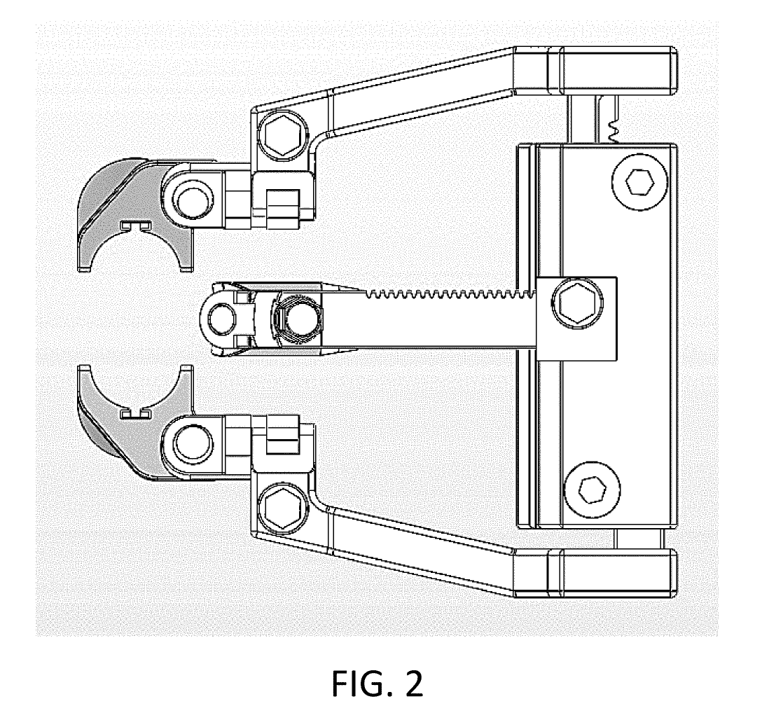

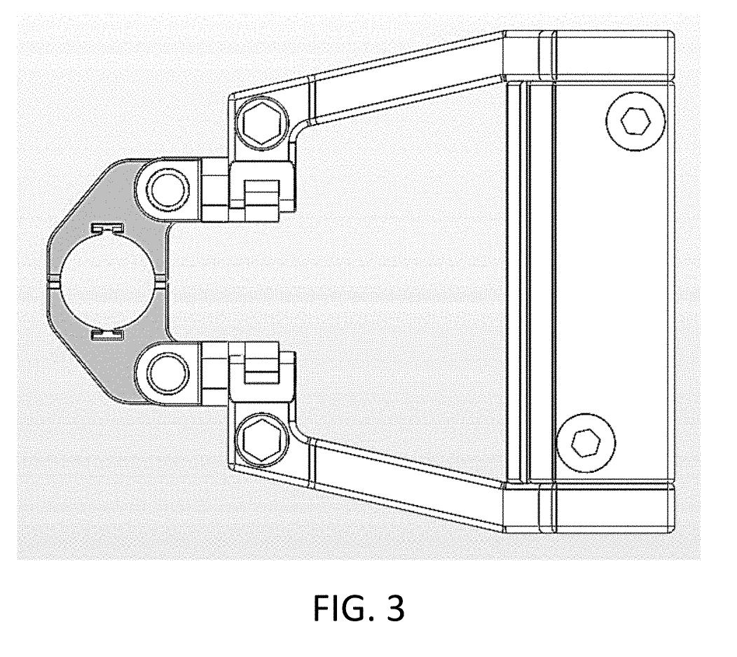

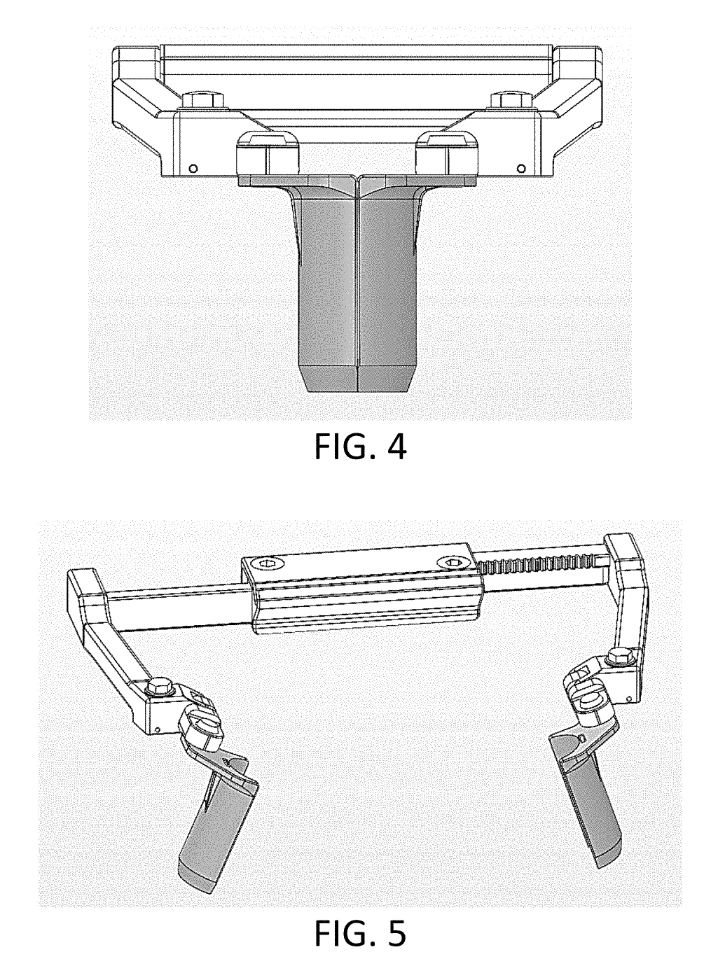

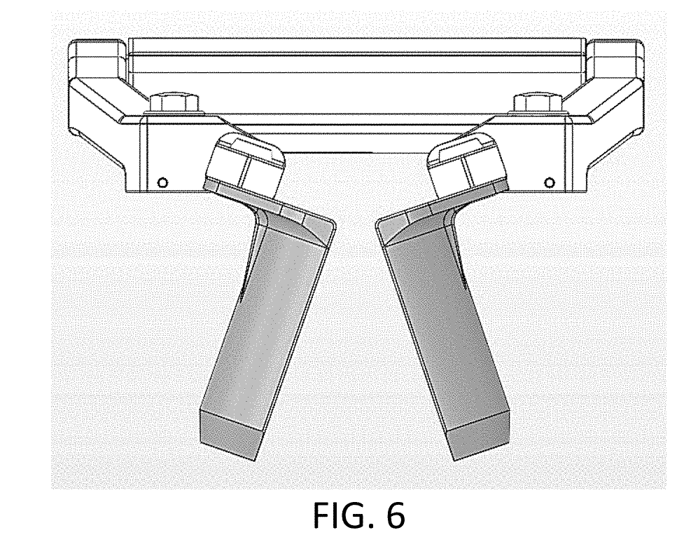

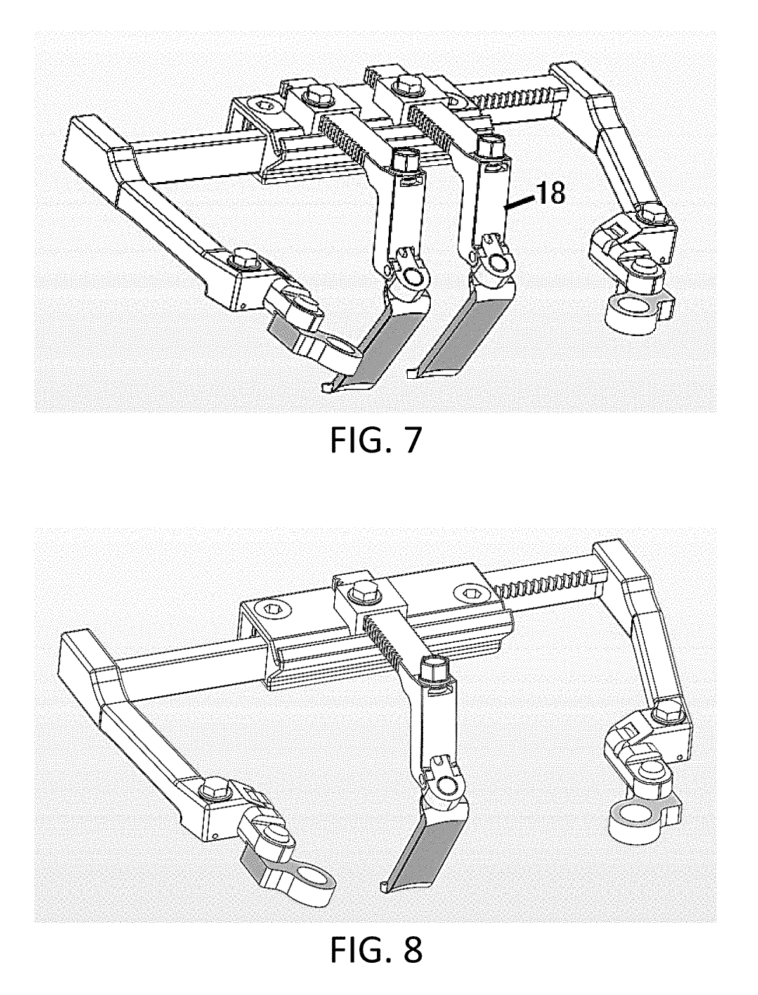

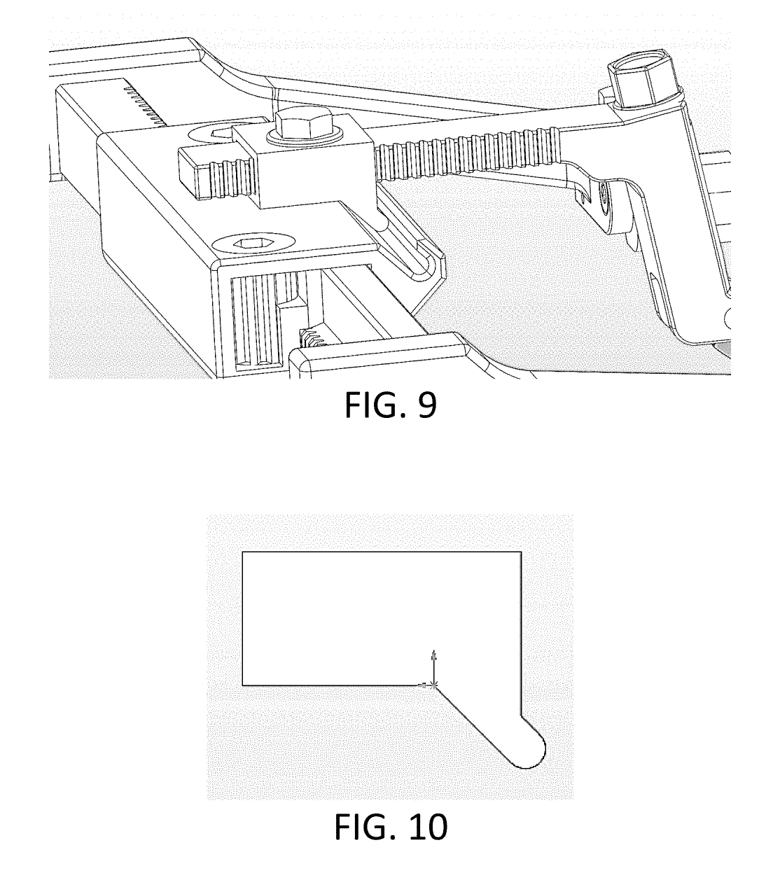

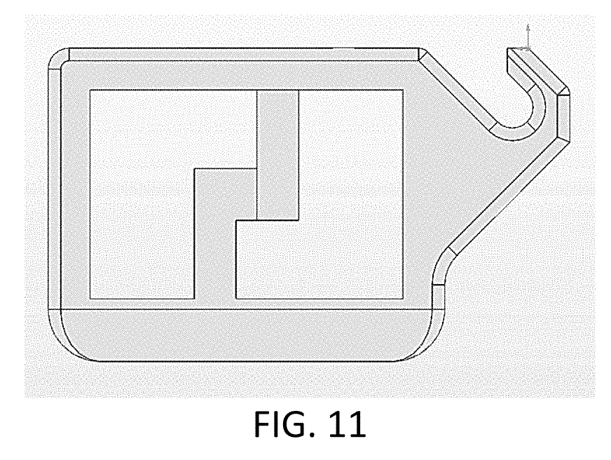

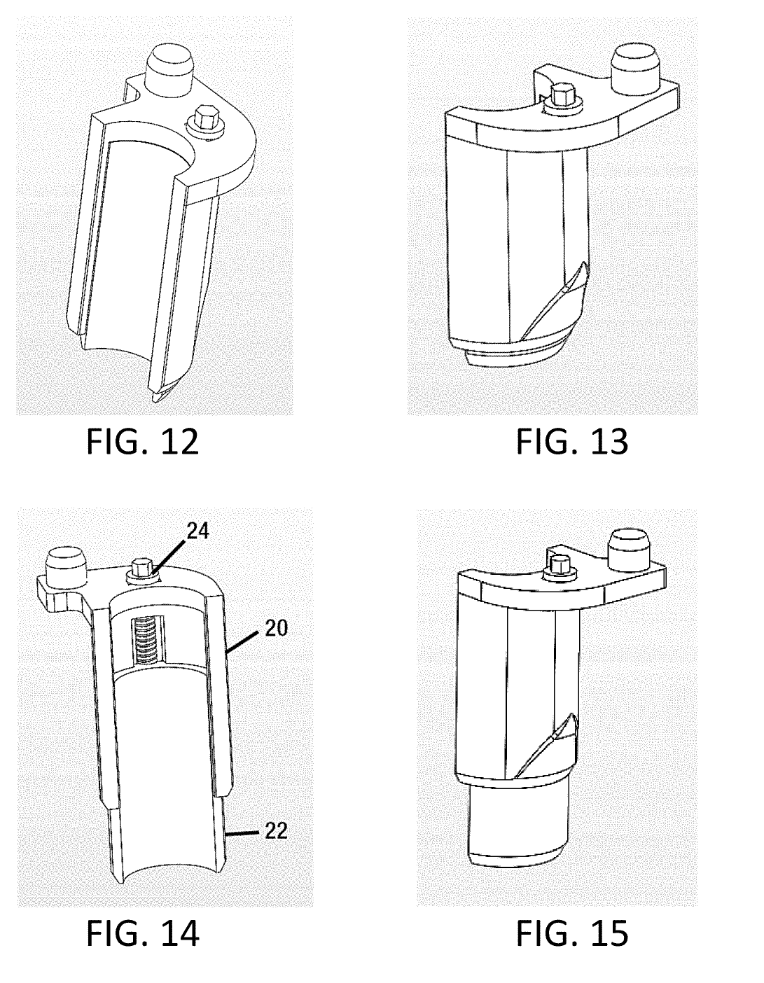

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

As can be seen throughout the figures, the retractor 10 includes a frame 11 and a toe adjuster 12 such that a user can adjust the toe and the blade length for optimal control.

The retractor also includes a middle blade assembly 14 which may be attached to the frame 11 in a quick connect slidably adjustable mechanism 16 such as a tongue and groove. More than one blade can be attached at this middle section. The medial or middle blade(s) 18 may be adjustable along the axis of the retractor frame 11 to accomplish or move cephalad or caudal relationship to the incision giving the user the ability to open the window of visibility as desired.

The adjustable blades may include a blade body 20 and an adjustable blade portion 22. The length of the blades may be adjusted using any desired mechanism including but not limited to a screw actuated mechanism 24, a spring loaded mechanism, a rack and pinion mechanism and/or a tensioning mechanism.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. A retractor, comprising:
a frame;
a toe adjuster provided to the frame; and
a blade secured to the toe adjuster.

2. The retractor of claim 1, wherein the blade comprises:
a blade body; and
an adjustable blade portion provided to the blade body,
wherein a blade length is adjustable by moving the adjustable blade portion longitudinally with respect to the blade body.
